(19) United States
(12) Patent Application Publication
(10) Pub. No.: US 2008/0095741 A1
(43) Pub. Date: Apr. 24, 2008

(54) R-(+)-alpha-liponic acid for the prevention of diabetes

(75) Inventors: Klaus Wessel, Bad Vilbel (DE); Chris Rundfeldt, Coswig (DE); Peter Ruus, Dittelsheim-Hessloch (DE)

Correspondence Address: VENABLE LLP, P.O. BOX 34385, WASHINGTON, DC, 20043-9998, US

(73) Assignee: MEDA Pharma GmbH & Co. KG, Homburg (DE)

(21) Appl. No.: 11/785,579

(22) Filed: Apr. 18, 2007

Related U.S. Application Data
(63) Continuation of application No. PCT/EP05/10927, filed on Oct. 11, 2005.

(30) Foreign Application Priority Data
Oct. 18, 2004 (DE) 102004050948.4

(51) Int. Cl. A61K 38/21; A61K 31/385; A61P 3/04

(52) U.S. Cl. 424/85.4; 514/440

(57) ABSTRACT

The invention relates to the use of R-(+)-α-lipoic acid in oxidised or reduced form or derivatives thereof alone or in combination with other effective compounds for the prevention of diabetes.

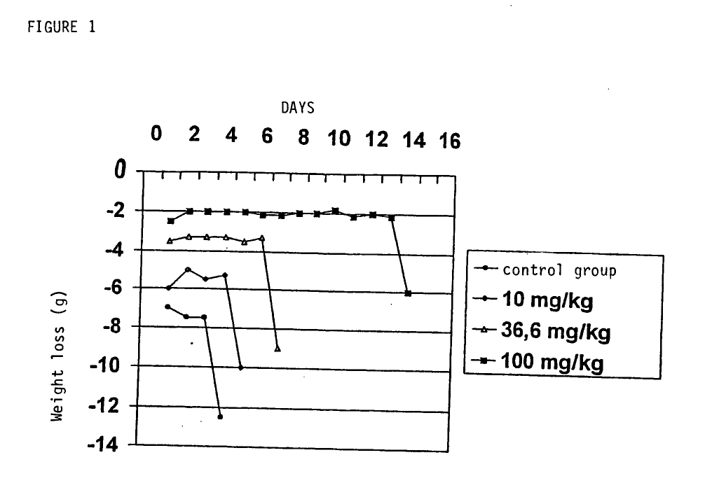

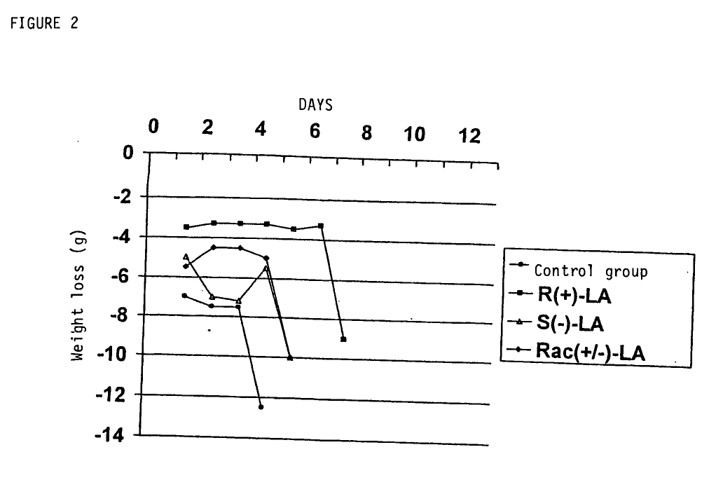

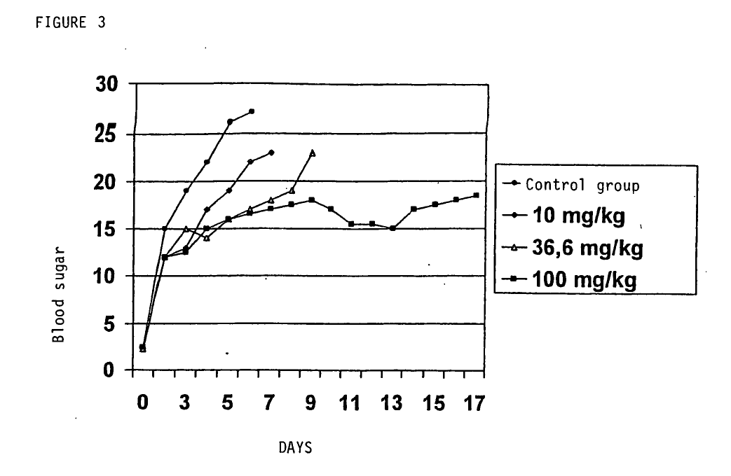

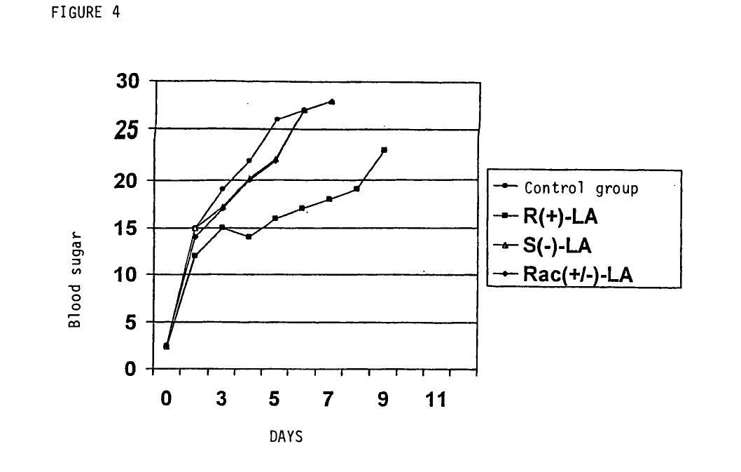

### TECHNICAL AREA

The invention relates to the use of R-(+)-α-lipoic acid in oxidized or reduced form or of its derivatives alone or in combination with other active compounds for the prevention of diabetes.

### STATE OF THE ART

R-(+)-α-lipoic acid (thioctic acid, 1,2-dithiolane-3-pentanoic acid) is a biological cofactor of α-keto acid dehydrogenases in the mitochondria and participates in the biooxidation of these acids (pyruvate, α-keto glutarate).

α-Lipoic acid in racemic form is used for the treatment of malaises in diabetic peripheral polyneuropathy as well as for the treatment of diseases of the liver or of fungal and metal poisonings.

The biological and therapeutic effects of α-lipoic acid in oxidized or reduced form are also found in numerous derivatives and metabolites of the oxidized or reduced base molecule in partially attenuated, partially amplified form (e.g., in 3-keto lipoic acid, 6-thioctic amide, octothiamine, 2-(N,N-dimethylamine) ethylamidolipoate HCL, tocopheryl- and tocotrienyl lipoate, γ-hydroxybutyrate lipoate, lipoic acid vitamin E ester, N-acetyl-p-aminophenol derivatives and others; see in this regard, e.g., Tirosch et al., Free Rad. Biol. Med. 26 (11/12), 1418-1426, 1999; EP 0855 396 A1; EP 0869126 A1; PCT/GB/98/02155; PCT WO 99/06040; DE 4327 462 A1 and PCT WO 00/24734). A number of, e.g., acyloxymethyl prodrugs from racemic lipoic acid including valeric acid and γ-linolenic acid have been described that have an intact dithiolane ring as common feature, coupled to a short-chain fatty acid (Redden et al., Int. J. Pharm. 180 151-160, 1999).

These derivatives, preferably derivatized on the carboxyl group or the mercaptan group, were suggested for improving the metabolism and the distribution in vivo. Individual derivatives can also improve the affinity and the conversion rate of α-lipoic acid on the biological target structures (biological redox systems such as α-keto acid dehydrogenases, H-protein, thioredoxin, glutathione reductase or cellular redox systems such as glutathione, ubiquinone, complexes of the respiratory chain, or redox- and SH-sensitive proteins and enzymes, the NO system, catalysis, the cellular cystine/cysteine shuttle, homocysteine, tyrosin kinase, MAP kinase, metallic ions for complex formation, alpha1-anti-proteinase, or redox-sensitive transcription factors such as NF-kB or AP1), or couple other active molecules to α-lipoic acid with the goal of synergistic or additive pharmacological action.

Therefore, the expression “R-(+)-α-lipoic acid” is used in the framework of this application as a general expression that also covers derivatives (esters, ethers, salts—in particular tromethamine—, amides, metabolites, etc.) to the extent the active dithiolane group of α-lipoic acid continues to be responsible for the biological and medicinal action of the derivative.

It was suggested, based on the antioxidant function of α-lipoic acid, that α-lipoic acid be used as a food supplement agent or as a preparation for medicinal nourishment alone or in combination with other substances (U.S. Pat. No. 5,569,670; U.S. Pat. No. 5,599,835). Food supplement agents for medicinal nourishment agents constitute product categories regulated according to certain national laws, within which the production and the marketing of products for certain health purposes in humans are regulated outside of the classic drug range.

α-Lipoic acid was furthermore suggested for treating circulatory disturbances in smokers and diabetics (U.S. Pat. No. 5,532,269) as well as a rheologic agent for diabetics (DE 4439477 C2).

It was furthermore determined that the R-(+) enantiomer of lipoic acid can be used with advantage in the combating of inflammatory diseases as well as in the cytoprotection of cells (EP 0382066 A2).

In addition, the R-(+) enantiomer of lipoic acid was suggested as a substance for treating insulin resistance and hyperglycemia in diabetes (DE 4343593 A1) since the reduced action of insulin on target cells (musculature, fat) can be re-normalized.

In the therapy of diabetes an improvement of the blood sugar level is aimed for. This can be achieved by an increased secretion of insulin from the β cells of the pancreas (sulfonyl ureas), by an exogenous insulin substitution, by a reduction of the glucose production of the liver (metformin) or, as presented above, by an improvement of the peripheral insulin action (thiozolidinedione, R-(+)-α-lipoic acid.

Diabetes is a disease that is characterized by a pathological elevation of the blood sugar (hyperglycemia)—in particular of the fasting blood sugar. Previously, patients with impaired blood sugar peaks without hyperglycemia at rest were also listed as pre-diabetics.

A distinction is made first of all between the main groups, in addition to other diabetes forms, of type 1 diabetes (juvenile diabetes) and type 2 diabetes (adult-onset diabetes). Untreated, diabetes results with increasing hyperglycemia in high water loss, weight loss and death. Other, rare forms of diabetes are, e.g., idiopathic diabetes, genetic β-cell defect forms, diseases of the endocrine pancreas, endocrinopathies, drug- or chemically induced diabetes, gestational diabetes, MODY diabetes or mitochondrial diabetes forms.

Whereas in the case of type 1 diabetes there is a lack of insulin due to a malfunction of the β cells that progresses usually rapidly in children and often slowly and progressively in adults and that must be treated sooner or later by insulin substitution and results up to a complete dependency on exogenous insulin substitution, type 2 diabetes can often be treated for a long time after diagnosis with oral anti-diabetic agents. Although type 2 diabetics often still have a high insulin secretion, the β-cell function is nevertheless involved. However, even in the case of adult-onset diabetes a loss of the function of the β-cell occurs and it must be substituted with the insulin.

The mechanisms of β-cell damage as the basis of the eruption of diabetes as well as the basis of the progressive deterioration of other diabetes forms are heterogeneous and only partially understood. However, in the case of juvenile diabetes a disturbance of the non-inflammatory, cellular immune response appears to be in the foreground, whereas in the case of adult-onset diabetes in particular the chronic stressing with high blood sugar levels contributes to the malfunction of the β cells. β cells in the pancreas are responsible in healthy humans for the physiological secretion of insulin and are limited in their function in diabetes. In addition, there are genetic factors, but even environmental toxins, drugs or customary activities in daily life can contribute to the occurrence and the progression of the loss of the β cell function.

### PRESENTATION OF THE INVENTION

In addition to the current symptom-oriented therapy (adjustment of the blood sugar) of manifest diabetes another medical goal is to prevent the occurrence of diabetes and its progression by intervention with suitable active substances. It is also an open therapeutic goal to delay the progressive deterioration of diabetes toward insulin dependency. These are the goals of a prevention, namely, the prevention or delaying of diabetes in patients with pre-diabetes as well as the delaying of the progression of manifest diabetes, especially the delaying of insulin dependency. Whereas pharmacological interventions have previously been totally lacking, patients base their hope on the transplantation of human β cells. Although great progress has been made here, a broadly applicable method of cytotransplantation is not yet available. Even given the availability of the method, biological material is available only for a fraction of the patients. For its part, β cell transplantations require protection against immunoreactions or creeping glucotoxicity.

In order to approach the goal of finding therapeutic concepts for the prevention of diabetes, the National Institute of Health in the United States has started the Diabetes Prevention Program (DPP) (WY [sic—W.Y.?] Fujimoto, Background and recruitment data for the US Diabetes Prevention Program, Diabetes Care 23 (2), B11-B13, 2000). Methods are to be tested in it that form the clinical basis for drug admissions in this new indication range.

17 million diabetics with approximately 800,000 new admissions per year are expected for the USA alone. The cost of diabetes in the USA is approximately 100 billion marks per year. More than 20 million patients have pre-diabetes and will sooner or later develop diabetes. Each therapeutic intervention with the goal of preventing or also even delaying the genesis of diabetes or of slowing it in its progression is extremely significant in the sense of conserving immense human and economic resources.

Since radical processes are suspected in the destruction of β cells, the influence of antioxidants on the glucose toxicity of β cells was examined. However, it turned out that classic antioxidants such as vitamin E and vitamin C have no effect but on the other hand the SH-containing antioxidant N-acetylcystein shows a certain protective action (Kaneto et al., 1999; Diabetes; 48 (12): 2398-406).

In further examinations isolated pancreatic cells were exposed to oxygen radical stress, either by a radical-generating system or by activated macrophages, that resulted in cellular lysis. It was found thereby that the pancreatic cells can be partially protected from lysis by pre-incubation with racemic dihydrolipoic acid (Burkart et al., Agents Actions 38, 60-65, 1993).

In further studies the NOD mouse was used as a model of type I diabetes in which the destruction of β cells takes place on the basis of immunological processes. The infiltration with immunocompetent cells into the pancreatic tissue concerned is viewed as a surrogate of the destruction of p cells. The chronic [long-term] administration of racemic α-lipoic acid had no influence on this surrogate. A protective action with racemic α-lipoic acid was not found until upon additional non-specific intoxication with a cytotoxin (cyclophosphamide) (Faust et al., Int. J. Immunopharmacol. 16 (1), 61-66, 1994). It was discussed that the observed effect of racemic α-lipoic acid is conditioned by anti-inflammatory or anti-radical or other mechanisms. However, it remained open in the structure of the experiments whether the effect against the endogenous loss of the β cells in the sense of a preventive action counteracted the intrinsic pathologic process or whether a non-specific protective effect against the additionally administered cytotoxin was observed. However, it must be assumed that the observed effect was brought about via the latter mechanism since no protection of the β cells was able to be demonstrated in the treatment group without the administration of cytotoxin. In addition, it has long been known that racemic α-lipoic acid attenuates the toxic effects of cytostatic agents (Fachinformation Thioctacid [German—Professional Information Thioctic Acid]; Berger et al., Arzneim.-Forsch./Drug Res. 33 (II) No. 9, 186-1288, 1983).

Therefore, the present invention had the problem of preparing an agent for the prevention of diabetes and the delaying of the loss of function of β cells after transplantation.

It was now found that the use of R-(+)-α-lipoic acid in oxidized or reduced form or its derivatives alone or in combination with other effective compounds results in the prevention of diabetes and the delaying of the loss of function of β cells after transplantation.

The experiments for the primary and secondary prevention of diabetes with α-lipoic acid or its derivatives were investigated using a suitable model system. The biobreed rat is a model of diabetes in which a primary loss of p cells also occurs on account of autoimmune processes. The model is superior to the NOD mouse model and closer to the human pathological situation in as far as in it the progression of diabetes can be followed even without the addition of non-specific cytotoxins. A secondary stress of the p cells occurs with the simultaneous development of hyperglycemia on account of the increasing glucose toxicity. Thus, pathogenic processes are reflected in this model that stand in the foreground of the progression of the disease and contribute to the progression in all forms of diabetes.

A preventive therapy with racemic α-lipoic acid as well as the two enantiomers took place in the biobreed rat.

It was now found that the occurrence of diabetes in the sense of prevention can be delayed and the progression of manifest diabetes can be distinctly attenuated in a dose-dependent manner with the R-(+)-enantiomer. The S-(−)- enantiomer and the racemic mixture remained without effect as concerns the occurrence of diabetes as well as what concerns its secondary progression. This allows the conclusion that for the primary and secondary prevention of diabetes only the R-configured, dextrorotary enantiomer is suitable, as well as derivatives derived from it with intact dithiolane structure. This can be used just as suitably for the prevention of the secondary loss of function of β cells after transplantation.

An explanation for the observed enantioselectivity of this effect can not yet be furnished since, as described above, SH reagents (N-acetylcystein) absolutely display effects in a similar sense, which allows the conclusion at first of a lacking enantioselectivity of the p cell protection in the case of lipoic acid, which, however, can not be the case in view of the results of the present invention.

The R-(+)-α-lipoic acid can be used in the scope of the invention in reduced or oxidized form even in the form of its derivatives such as salts, amides, esters, ethers or metabolites. The tromethamine salt of R-(+)-α-lipoic acid is especially preferred.

R-(+)-α-lipoic acid or derivatives can be manufactured in oral forms of administration (tables or capsules with rapid or delayed release, drinking solutions) or parenterally, intravenously or intramuscularly from ampoules or ready infusions in accordance with the general state of the art as it is described, e.g., in the following cited documents: EP 0858 802 A2, EP 0318 891 A1, EP 0 560 092 B1, U.S. Pat. No. 5,650,429 A, U.S. Pat. No. 5,334,612 A, U.S. Pat. No. 5,569,670 A, U.S. Pat. No. 9,755,433 A, PCT/US 99/11178, PCT WO 00/24734, U.S. Pat. No. 6,191,162 B1 and WO 9840053.

The products manufactured in this manner can be brought labeled into the market for the purpose of being used in the area of application for the prevention of diabetes while taking into consideration the appropriate national regulations for the instructions of use for professional personnel and the patient. The products can customarily be considered in this case in the legal framework of drugs or, if applicable, of food supplement substances.

According to an embodiment of the present invention in order to improve the efficacy or compatibility of R-(+)-α-lipoic acid in oxidized or reduced form or its derivative, a free or fixed combination or applicable composition with one or several effective substances can be used that are selected from the group of vitamin C and derivatives, vitamin E and tocopherols, ubiquinone, vitamin B1-B12, thiamin and riboflavin, pantothenic acid, biotin, inositol, β-carotenes, zinc, magnesium, selenium, taurin, choline, N-acetylcystein and other cysteines, unsaturated fatty acids, essential fatty acids of the n-3 and n-6 series, γ-linolenic acid and derivatives, aspirin and other non-steroidal antiphlogistics, steroids, L-carnitine and other derivatives.

According to an embodiment of the invention the component of a fixed or free combination of applicable compound of R-(+)-α-lipoic acid can be an oral antidiabetic agent, e.g., a sulfonyl urea, optionally with other active substances in order to preventively counteract diabetes.

In order to further elevate the use of an administration of R-(+)-α-lipoic acid in oxidized or reduced form or its derivatives, an individual therapy regime is indicated that can comprise diet and sports activity as well as the preventive administration of properly dosed antidiabetic agents, insulin, analogues or mimetics. Also, the administration of lipid lowerers, blood-pressure agents and coronary circulation preparations, especially ACE inhibiters, but also other vasoactive substances can be a component of the therapy in free or fixed combination for the therapy.

The invention is explained in detail in the following using examples and diagrams.

### EXAMPLE 1

Biobreed rats (male and female, 130-180 g) 5-19 weeks old were chronically treated (ip) with different doses of R-(+)-α-lipoic acid as tromethamine salt. The development and progression of diabetes was determined by weight loss as surrogate. It turned out that the animals developed a delayed progression of diabetes in a dose-dependent manner.

### EXAMPLE 2

Biobreed rats (male and female, 130-180 g) 5-19 weeks old were each chronically treated (ip) with 36.6 mg of different lipoic acid derivatives (as tromethamine salt). The development and progression of diabetes was determined by daily weight measuring as surrogate. It turned out that only the R-configured derivative had an effect on the progression of diabetes.

### EXAMPLE 3

Biobreed rats (male and female, 130-180 g) 5-19 weeks old were chronically treated (ip) with different doses of R-(+)-α-lipoic acid as tromethamine salt. The development and progression of diabetes was determined by daily measuring of the blood sugar (taken from the caudal vein) as surrogate. It turned out that the animals developed a delayed progression of a rise in blood sugar in a dose-dependent manner.

### EXAMPLE 4

Biobreed rats (male and female, 130-180 g) 5-19 weeks old were each chronically treated with 36.6 mg of different lipoic acid derivatives (as tromethamine salt (ip)). with as tromethamine salt. The development and progression of diabetes was determined by daily measuring of the blood sugar (taken from the caudal vein) as surrogate. It turned out that only the dextrorotary derivative had an effect on the progression of diabetes.

The survival time and the water consumption were determined as further surrogates for the occurrence and deterioration of diabetes. A positive effect of the R-configured derivative of α-lipoic acid was displayed in a dose-dependent manner even in the survival time and in the increase of the consumption of water whereas the other entities tested remained ineffective.

### EXAMPLE 1

Days

[See page 12 for graphic and data requiring no translation.]

Weight loss (g) Control group

### EXAMPLE 2

Days

[See page 13 for graphic and data requiring no translation.]

Weight loss (g) Control group

### EXAMPLE 3

[See page 14 for graphic and data requiring no translation.]

Blood sugar (mmol/ml) Control group

Days

### EXAMPLE 4

[See page 15 for graphic and data requiring no translation.]

Blood sugar (mmol/ml) Control group

Days

1-7. (canceled)

8. A method of preventing diabetes in an individual, said method comprising administering an effective amount of R-(+)-α-lipoic acid in oxidized or reduced form, or a derivative thereof, to said individual.

9. The method of claim 8, wherein the derivative is selected from the group consisting of a salt, an amide, an ester, an ether and a metabolite.

10. The method of claim 8 wherein said individual is human.

11. The method of claim 8, additionally comprising the administration of a substance selected from the group consisting of vitamin C and derivatives, vitamin E and tocopherols, ubiquinone, vitamin B1-B12, thiamin, riboflavin, pantothenic acid, biotin, inositol, β-carotene, zinc, magnesium, selenium, taurin, choline, N-acetylcystein and derivatives, unsaturated fatty acids, essential fatty acids of the n-3 and n-6 series, γ-linolenic acid and derivatives, aspirin and non-steroidal antiphlogistics, steroids, immunosuppressants such as interferon, L-carnitine and derivatives.

12. The method of claim 8, additionally comprising an individual therapy regime including one or more components selected from the group consisting of diet, exercise, and the administration of an antidiabetic agent, lipid reducing agent, blood-pressure reducing agent, coronary circulation preparation, or vasoactive substance.

13. The method of claim 12, wherein an ACE inhibitor is administered.

14. A method of delaying the loss of function of β cells after transplantation in an individual, said method comprising administering an effective amount of R-(+)-α-lipoic acid in oxidized or reduced form or a derivative thereof, to said individual.

15. The method of claim 14, wherein the derivative is selected from the group consisting of a salt, an amide, an ester, an ether and a metabolite.

16. The method of claim 14 wherein said individual is human.

17. The method of claim 14, additionally comprising the administration of a substance selected from the group consisting of vitamin C and derivatives, vitamin E and tocopherols, ubiquinone, vitamin B1-B12, thiamin, riboflavin, pantothenic acid, biotin, inositol, β-carotene, zinc, magnesium, selenium, taurin, choline, N-acetylcystein and derivatives, unsaturated fatty acids, essential fatty acids of the n-3 and n-6 series, γ-linolenic acid and derivatives, aspirin and non-steroidal antiphlogistics, steroids, immunosuppressants such as interferon, L-carnitine and derivatives.

18. The method of claim 14, additionally comprising an individual therapy regime including one or more components selected from the group consisting of diet, exercise, and the administration of an antidiabetic agent, lipid reducing agent, blood-pressure reducing agent, coronary circulation preparation, or vasoactive substance.

19. The method of claim 18, wherein an ACE inhibitor is administered.

20. A pharmaceutical composition comprising R-(+)-α-lipoic acid in oxidized or reduced form, or a derivative thereof, and at least one substance selected from the group consisting of vitamin C and its derivatives, vitamin E and tocopherols, ubiquinone, vitamins B1-B12, thiamin, riboflavin, pantothenic acid, biotin, inositol, β-carotene, zinc, magnesium, selenium, taurin, choline, N-acetylcystein and derivatives, unsaturated fatty acids, essential fatty acids of the n-3 and n-6 series, γ-linolenic acid and derivatives, aspirin and non-steroidal antiphlogistics, steroids, L-carnitine and derivatives.

21. The pharmaceutical composition of claim 20, wherein the derivative is selected from the group consisting of a salt, an amide, an ester, an ether and a metabolite.

22. The pharmaceutical composition of claim 20, additionally comprising an agent selected from the group consisting of an oral antidiabetic agent, an immunosuppressive agent, a lipid reducing agent, a blood-pressure agent, a coronary circulation preparation.

23. The pharmaceutical composition of claim 22 that comprises a vasoactive substance.

24. The pharmaceutical composition of claim 23 wherein said vasoactive substance is an ACE inhibitor.

25. The pharmaceutical composition of claim 22 that comprises an immunosuppressive agent.

26. The pharmaceutical composition of claim 25, wherein said immunosuppressive agent is an interferon.
